(19) United States
(12) Patent Application Publication
(10) Pub. No.: US 2013/0053676 A1
(43) Pub. Date: Feb. 28, 2013

(54) METHOD AND SYSTEM FOR REDUCING PATIENT MOTION DURING MEDICAL DIAGNOSTIC IMAGING

(71) Applicants: ERIK PAUL KEMPER, Franklin, WI (US); RYAN RAMOS, Greenfield, WI (US)

(72) Inventors: ERIK PAUL KEMPER, Franklin, WI (US); RYAN RAMOS, Greenfield, WI (US)

(73) Assignee: GENERAL ELECTRIC COMPANY, SCHENECTADY, NY (US)

(21) Appl. No.: 13/215,454

(22) Filed: Aug. 23, 2011

(51) Int. Cl. A61B 5/05

(52) U.S. Cl. 600/407

(57) ABSTRACT

A medical imaging system includes a gantry having a bore extending therethrough, a bore cover disposed within the bore, and an element disposed on the bore cover, the element being viewable by a patient positioned within the bore to reduce patient motion. A method of imaging a patient is also described herein.

### BACKGROUND OF THE INVENTION

The subject matter herein relates generally to medical imaging systems, and more particularly, to a method and system for reducing an anxiety level of a patient and thus enhancing patient image quality.

Medical imaging systems exist that scan patients to obtain medical information. Conventional imaging systems are typically installed in a room that is outfitted for the particular medical imaging system being utilized. For example, a conventional computed tomography (CT) imaging system may be installed in a dedicated CT imaging system room. The conventional CT imaging system includes a gantry that defines an opening or bore therethrough. To image the patient, the patient is positioned on a table and then moved within the bore where imaging is performed.

In operation, conventional medical imaging systems may exhibit image quality that is affected by motion of the patient being imaged. For example, patient motion may result in image artifacts in the final image. To reduce the patient motion during an imaging procedure, the imaging system operator typically instructs the patient to remain substantially motionless while the imaging information is being acquired. However, involuntary motion encountered in medical imaging systems is a common source of image artifacts. The involuntary motion may be lead to errors, such as when a physician is determining the size of a lesion, determining the location of the lesion, or quantifying the lesion.

Involuntary motion may be caused by numerous physiological parameters. For example, breathing motion, motion of the heart, e.g. accelerated heart rate, and/or involuntary motion of a head, arm, or leg of the patient. In some instances, being positioned within the relatively small imaging system bore may cause the heart rate or breathing of the patient to increase resulting in involuntary motion.

For example, patients are generally unfamiliar with the imaging system and the surrounding environment. More specifically, while medical personnel are accustomed to the rather sterile environment surrounding the medical imaging system, being positioned within the bore of the medical imaging system may increase the anxiety level of the patient being imaged. Moreover, the bore of the conventional imaging system does not include any device to enable the patient to focus their attention while the imaging information is being acquired. Accordingly, at least some patients may choose to either close their eyes or search a blank surface of the bore for some item of interest.

Conventional external systems include, for example, a headset worn by the patient during imaging or an external device. Optionally, an external system may be worn by the patient during imaging. However, the conventional headsets may cause discomfort to the patient resulting in an increase in the anxiety level that results in patient motion. Additionally, the external system may interfere with the patient being positioned on the table for imaging. Moreover, the conventional systems may result in an increase in the cost of the imaging system. Therefore, because conventional imaging systems do not include a relatively inexpensive and easy to utilize device to focus the patient's attention, the patient's attention may be focused on the length of the examination procedure and/or uncomfortable physical conditions resulting in increased patient anxiety. The increased patient anxiety may cause an increase in the some of the patient's physiological parameters such as the patient's breathing or the patient's heart rate, resulting in increased patient movement during imaging.

### BRIEF DESCRIPTION OF THE INVENTION

In one embodiment, a medical imaging system is provided. The medical imaging system includes a gantry having a bore extending therethrough, a bore cover disposed within the bore, and an element disposed on the bore cover, the element being viewable by a patient positioned within the bore to reduce patient motion.

In another embodiment, a method of imaging a patient is provided. The method includes selecting a theme based on the potential anxiety level, wherein the theme is presented in a manner to reduce a motion of the patient being imaged, attaching the theme to a bore cover of the medical imaging system, and performing a medical imaging scan of the patient using the medical imaging system.

### BRIEF DESCRIPTION OF THE DRAWINGS

FIG. 1 is a perspective view of an exemplary computed tomography (CT) imaging system formed in accordance with various embodiments.

FIG. 2 is a perspective view of a portion of the imaging system shown in FIG. 1 formed in accordance with various embodiments.

FIG. 3 is a plan view of an exemplary theme that may be utilized with the imaging system shown in FIG. 1 in accordance with various embodiments.

FIG. 4 is a perspective view of a portion of the imaging system shown in FIG. 1 formed in accordance with various embodiments.

FIG. 5 is a plan view of an exemplary theme that may be utilized with the imaging system shown in FIG. 1 in accordance with various embodiments.

FIG. 6 is a plan view of an exemplary theme that may be utilized with the imaging system shown in FIG. 1 in accordance with various embodiments.

FIG. 7 is a plan view of an exemplary theme that may be utilized with the imaging system shown in FIG. 1 in accordance with various embodiments.

FIG. 8 is a plan view of an exemplary theme that may be utilized with the imaging system shown in FIG. 1 in accordance with various embodiments.

FIG. 9 is a plan view of an exemplary theme that may be utilized with the imaging system shown in FIG. 1 in accordance with various embodiments.

FIG. 10 is a plan view of an exemplary theme that may be utilized with the imaging system shown in FIG. 1 in accordance with various embodiments.

FIG. 11 is a plan view of an exemplary theme that may be utilized with the imaging system shown in FIG. 1 in accordance with various embodiments.

FIG. 12 is a plan view of an exemplary theme that may be utilized with the imaging system shown in FIG. 1 in accordance with various embodiments.

FIG. 13 is a flowchart illustrating an exemplary method of imaging a patient in accordance with an embodiment of the present invention.

### DETAILED DESCRIPTION OF THE INVENTION

The foregoing summary, as well as the following detailed description of certain embodiments of the present invention, will be better understood when read in conjunction with the appended drawings. To the extent that the figures illustrate diagrams of the functional blocks of various embodiments, the functional blocks are not necessarily indicative of the division between hardware circuitry. Thus, for example, one or more of the functional blocks (e.g., processors or memories) may be implemented in a single piece of hardware (e.g., a general purpose signal processor or a block of random access memory, hard disk, or the like). Similarly, the programs may be stand alone programs, may be incorporated as subroutines in an operating system, may be functions in an installed software package, and the like. It should be understood that the various embodiments are not limited to the arrangements and instrumentality shown in the drawings.

As used herein, an element or step recited in the singular and proceeded with the word “a” or “an” should be understood as not excluding plural of said elements or steps, unless such exclusion is explicitly stated. Furthermore, references to “one embodiment” of the present invention are not intended to be interpreted as excluding the existence of additional embodiments that also incorporate the recited features. Moreover, unless explicitly stated to the contrary, embodiments “comprising” or “having” an element or a plurality of elements having a particular property may include additional such elements not having that property.

Various embodiments described herein are configured to reduce the motion of the patient while the patient is being imaged. Specifically, various embodiments described herein include themes that are specifically designed to reduce the motion level in patients including younger patients. The imaging system includes a theme that represents a place, a pattern, or other design that is readily apparent to the patient being imaged. The themes provide a distraction to focus the patient's attention during the imaging. The distraction therefore reduces the patient's focus on the imaging procedure itself and therefore reduces the patient's anxiety level during the imaging procedure. As a result, motion of the patient may be reduced, thereby decreasing the time required to perform imaging and improving image quality.

It should be noted that although the various embodiments are described in connection with computed tomography (CT) imaging system having particular components, the various embodiments are not limited to CT imaging systems. Accordingly, the various embodiments may be implemented in connection with any type of diagnostic imaging system, for example, medical diagnostic imaging system (e.g., a magnetic resonance imaging (MRI) system, a positron emission tomography (PET) imaging system, an ultrasound imaging system, an X-ray imaging system, and/or a single photon emission computed tomography (SPECT) imaging system, among others.

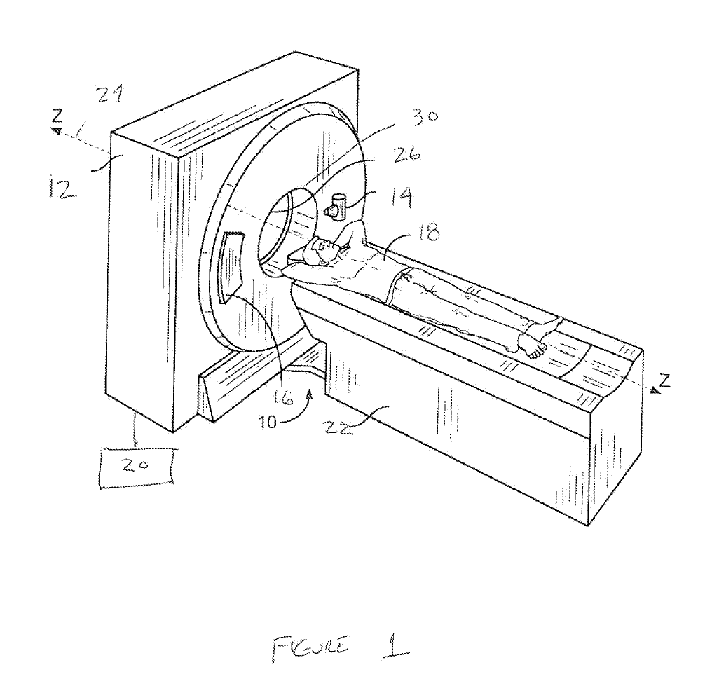

FIG. 1 is a pictorial view of an exemplary medical imaging system 10. In the exemplary embodiment, the medical imaging system 10 shown in FIG. 1 is a CT imaging system 10 that includes a gantry 12. The imaging 10 also includes an X-ray source 14 and a detector 16 that are mounted to the gantry 12. The detector 16 includes a plurality of detector elements (not shown), that are arranged in rows and channels, that together sense projected X-rays, from the X-ray source 14, that pass through an object, such as a patient 18. Each detector element produces an electrical signal, or output, that represents the intensity of an impinging X-ray beam and hence allows estimation of the attenuation of the beam as the beam passes through the patient 18. The imaging system 10 also includes a computer 20 that receives the projection data from the detector 16 and processes the projection data to reconstruct an image of the object 18.

The CT imaging system 10 also includes an imaging table 22 that is adapted to move the patient 18 with respect to the imaging unit 10. The imaging table 22 is configured to facilitate ingress and egress of the patient 18 into an examination position that is substantially aligned with an examination axis 24. During an imaging scan, the imaging table 22 may be controlled to move axially into and out of a bore 26 that is defined by the gantry 12. In the exemplary embodiment, the bore 26 is covered by a bore cover 30 that is fabricated using a substantially X-ray transparent material.

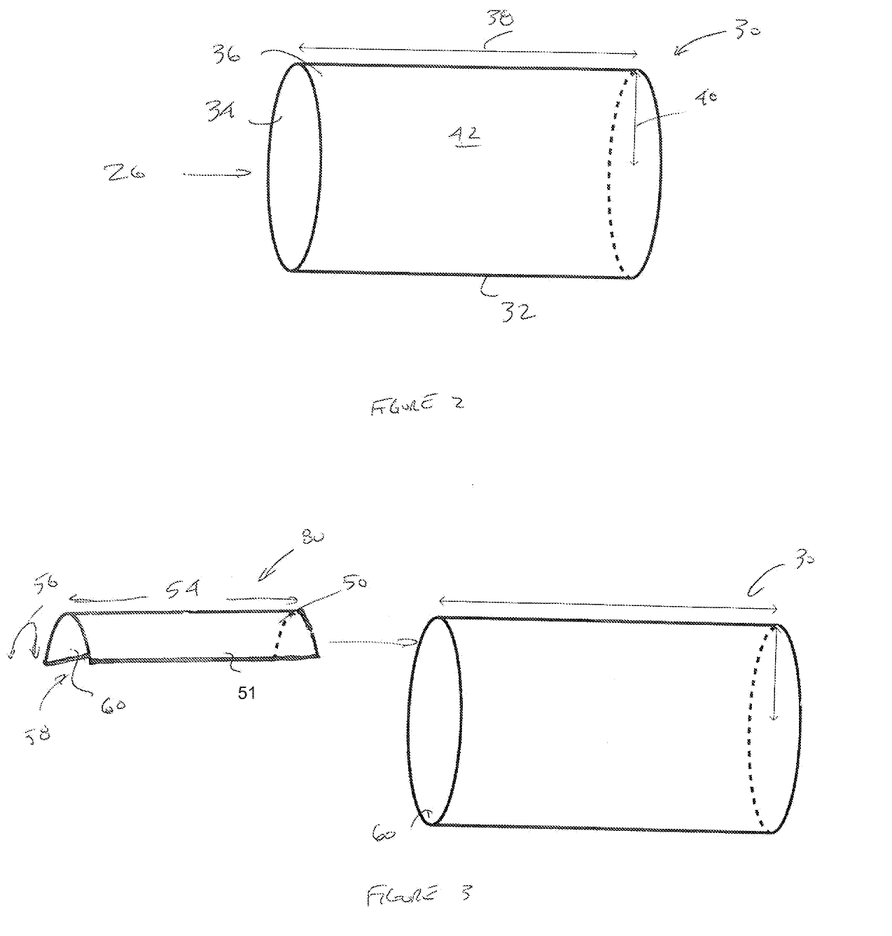

FIG. 2 is a perspective view of the exemplary bore cover 30 shown in FIG. 1 and formed in accordance with various embodiments. The bore cover 30 is defined by a material 32 that is formed to have a substantially cylindrical shape having an interior surface 34 that is disposed proximate the bore 26 and an exterior surface 36. Thus, the bore 26 also has a substantially cylindrical shape. The bore cover 30 may be formed as a unitary structure that is inserted into, and coupled to, the gantry 12. Optionally, the bore cover 30 may include a plurality of components that are coupled together and then inserted into, and coupled to the gantry 12. Thus the bore cover 30 has a length 38 and a radius 40. The length 38 and the radius 40 define a volume 42 that is formed by the bore cover 30 and represents the bore 26 of the imaging system 10.

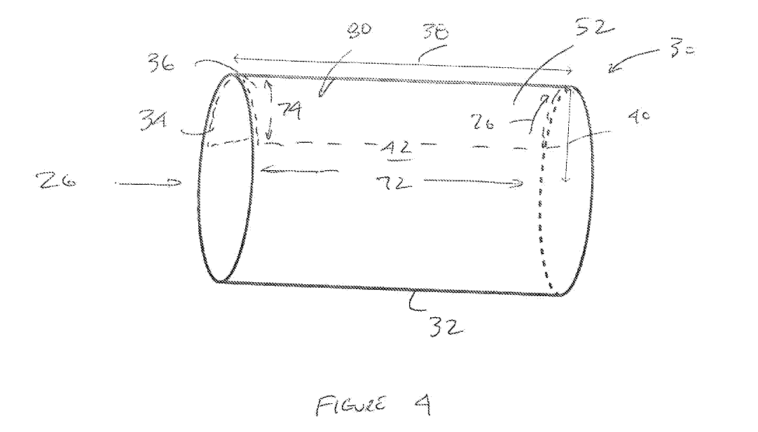

FIG. 3 is a plan view of an exemplary theme 50 that may be attached to the imaging system 10. In operation, the theme 50 forms a physical environment at least partially surrounding the patient 18. The theme 50 is configured to reduce an anxiety level of the patient 18 being imaged and therefore reduce patient motion. More specifically, as discussed above, conventional imaging systems may cause a patient to experience a vague unpleasant emotion that is experienced in anticipation of the medical examination. To facilitate reducing the patient's motion, and thus improve the image quality, the imaging system 10 is modified to have the theme 50. A theme, as used herein, represents an idea or subject that is physically implemented in the imaging system bore 26 to reduce patient motion. For example, some exemplary themes may include a paisley theme, a cloud theme, and/or a wood grain theme. Moreover, the theme 50 may be related to a special character or medical imaging system that is specifically utilized in a medical image environment to reduce the patient's motion. For example, an imaging system equipped to image small children may have a different theme than an imaging system equipped to image adults. Moreover, the themes may be interchangeable to enable a different theme to be installed on the same imaging system based on the specific patient being imaged.

In one embodiment, the theme 50 may be formed on a substrate 51 as shown in FIG. 3. The substrate 51 is preferably fabricated from an X-ray transparent material. In operation, the substrate 51, including the theme 50, may be removably coupled to the interior surface 34 of the bore cover 30 to enable different themes to be utilized with a single imaging system 10. For example, a first theme may be installed on the bore cover 30 to reduce the motion of a first patient and a second different theme may be installed on the bore cover 30 to reduce the motion of a different second patient.

The substrate 51, including the theme 50, has a length 54 and a width 56. The substrate 51 is formed to have a radius of curvature 58 that is substantially the same as a radius of curvature 60 of the bore cover 30. Accordingly, when the substrate 51 is coupled to the bore cover 30, the substrate 30 fits substantially flush to the interior surface 34 of the bore cover 30. In one embodiment, the length 54 of the theme 50 is substantially the same as the length 38 of the bore cover 30 to enable the patient 18 to view the theme 50 in any axial position within the bore 26 of the imaging system 10. Moreover, in the exemplary embodiment, the width 56 of the theme 50 is substantially less than the circumference of the bore cover 30. For example, assuming that the patient's field of view is approximately 120 degrees, the width 56 of the theme 50 may be selected to be less than 120 degrees to enable the patient being imaged to view the edges of the theme during the imaging procedure. Enabling the patient to view the edges of the theme 50 may facilitate reducing patient motion by providing the patient with a defined pattern within the patient's field of view for the patient to focus on.

In the exemplary embodiment, the theme 50 is also formed from a substantially X-ray transparent material. Accordingly, the theme 50 may be formed by depositing or printing ink onto an interior surface 60 of the substrate 51. Optionally, the theme 50 may be printed onto an appliqué. An appliqué, as used herein defines a device that is applied to another surface. For example, the appliqué may be formed to include the theme 50. The appliqué may then be attached to the substrate 51 using fasteners, or an adhesive, for example.

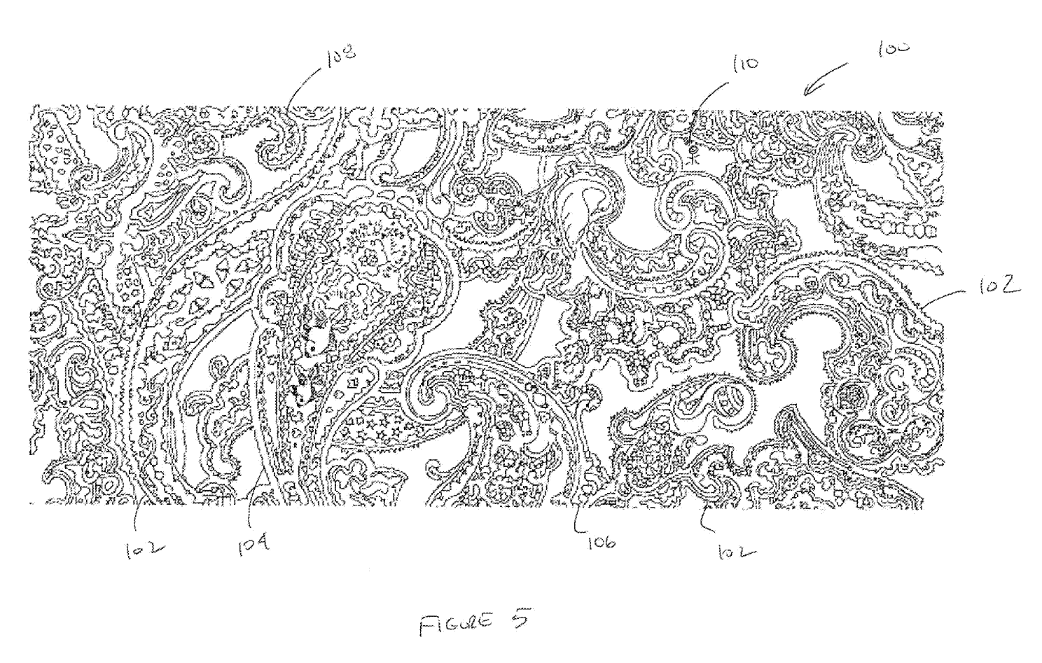

FIG. 4 is a plan view of an exemplary theme 52 that may be attached to the imaging system 10. In the exemplary embodiment, the theme 52 is formed directly on the interior surface 34 of the bore cover 30. In one embodiment, the theme 52 may be formed on the bore cover 30 using an X-ray transparent material. In the exemplary embodiment, a length 72 and a width 74 of the theme 52, when installed directly on the bore cover 30, is substantially the same as the length 54 and the width 56 of the theme installed on the substrate 51, as discussed above. Thus, when theme 52 is formed directly on the bore cover 30, the theme 50 has a radius of curvature 76 that is substantially the same as a radius of curvature 60 of the bore cover 30.

In the exemplary embodiment, the theme 52 is also formed from a substantially X-ray transparent material. Accordingly, the theme 52 may be formed by depositing or printing ink directly onto the interior surface 34 of the bore cover 30. Optionally, the theme 52 may be etched into the bore cover 30. In the exemplary embodiment, the theme 52 is formed unitarily with the bore cover 30 prior to the bore cover 30 being installed into the imaging system 10.

In the exemplary embodiment, the themes 50 and 52 are implemented using at least one element 80. The element(s) 80 represent physical modifications made to the medical imaging system 10 to provide a visual indication of the theme. For example, some physical modifications may include, as discussed above, an element 80 that is an appliqué that includes the theme 50, which is coupled to the substrate 51. Another element 80 may be an appliqué that is coupled directly to the interior surface 34 of the bore cover 30. A further element 80 may be the bore cover 30 which includes the theme 52 formed unitarily with, and directly onto the interior surface 34 of the bore cover 30. As such, the elements 80 represent physical modifications to the existing medical imaging system 10. The elements 80 may also form a pleasing or soothing pattern.

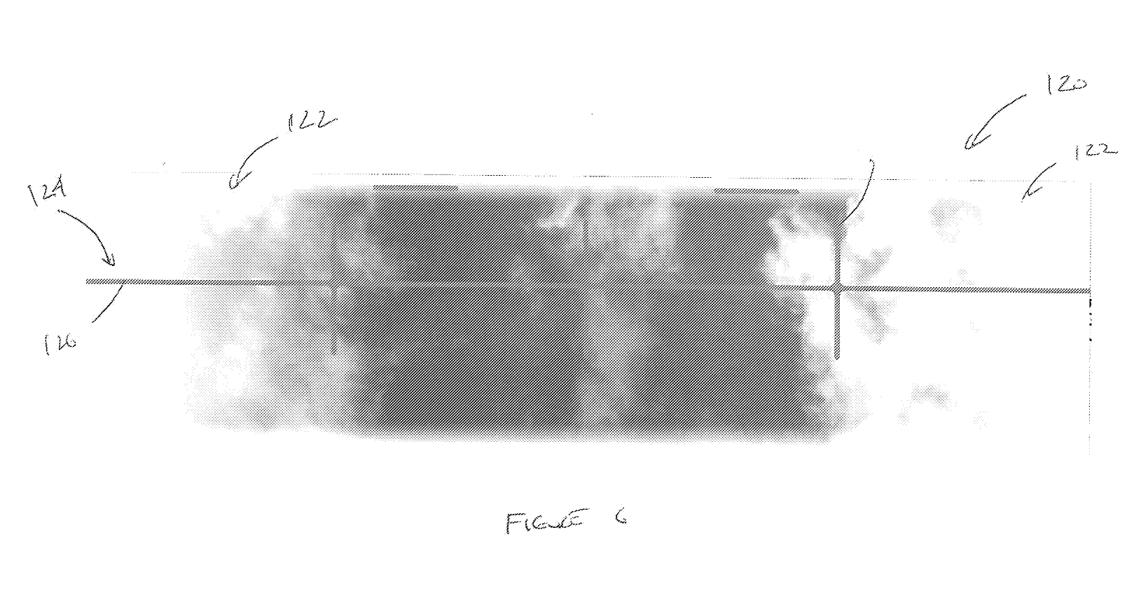

Referring to FIG. 5, the elements 80 may be embodied to include a theme 100. The theme 100 may be formed from a plurality of lines and colors that are applied to at least a portion of the imaging system 10. In the exemplary embodiment, the theme 100 is selected to reduce patient motion. More specifically, the theme 100 may be selected to mimic the patterns observed in the forest and/or in nature. Moreover, the theme 100 may be selected to include geometric patterns that are non-repeating to reduce vertigo and other effects that may cause the patient motion.

In the exemplary embodiment, the theme 100 is a paisley theme that is designed using a plurality of lines 102. The lines 102 may include lines 104 having a first thickness and lines 106 have a second thickness that is different than the lines 104. In general, thicker lines 104 are more easily observed by the patient 18, whereas thinner lines 106 require the patient 18 to focus more on the theme 100. Accordingly, the patient 18 focuses less on the imaging procedure. It should be realized the weights and shapes of the lines 102 may be adjusted to generate a theme that is most beneficial in reducing the patient's motion. Thus, the lines 102 may be continuous, have a darker or lighter shade, and may be thicker or thinner. Moreover, the theme 100 may include shading 108 that is provided between at least a portion of the lines 102. The shading 108 may have any hue or color that facilitates reducing patient motion. For example, both the lines 102 and the shading 108 may be formed using a lighter shade or hue to promote calmness in the patient. Additionally, the theme 100 may include one or more random design elements 110 that are utilized to focus the patient's attention. For example, the random design element 110 may be embodied as a picture of a person, or other object. In operation, the patient is focused on locating the design element 110 instead of the imaging procedure itself.

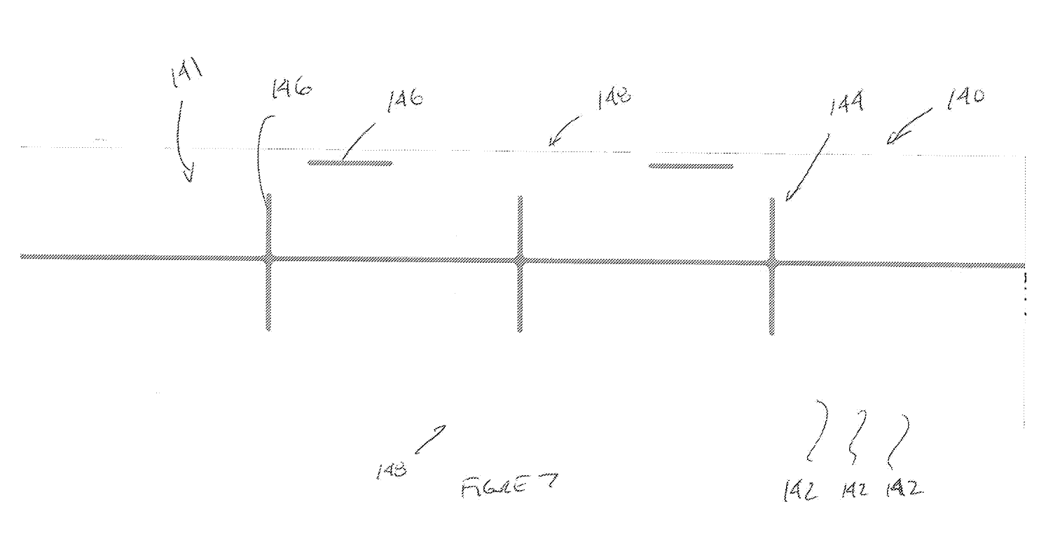

Referring to FIG. 6, elements 80 may be embodied to include a theme 120. The theme 120 may be a scenic design. A scenic design, as used herein, generally means a background “picture” that provides a physical appearance that represents the selected theme. For example, in the exemplary embodiment, the theme illustrated in FIG. 6 is a “Cloud Theme.” As such, the cloud theme 120 includes a visual representation of a plurality of clouds 122 that form the background of the cloud theme 120. The cloud theme 120 may also include a geometric pattern 124 that is formed using a plurality of lines 126.

In operation, the lines 126 allow the laser lights to clearly go through the pattern 124. The lines 126 may include lines having a first thickness and lines having a second thickness. Optionally, the lines 126 may all have the same thickness. It should be realized the weights and shapes of the lines 126 may be adjusted to generate a theme that is most beneficial in reducing the patient's motion. Thus, the lines 126 may be continuous, have a darker or lighter shade, and may be thicker or thinner. Moreover, in the exemplary embodiment, the lines 126 may be overlayed on at least a portion of the clouds 122. More specifically, the clouds 122 function as the background of the cloud theme 120 and the lines 126 function as the foreground of the cloud theme 120 such that the patient first observes the clouds 122 and then upon further visual inspection observes the lines 126 overlayed on the clouds 122. The lines 126 may be the same color as a portion of the clouds 122 or may be a different color. In operation, the patient's focus is therefore directed to first visually inspect the clouds and then visually inspect the size, shape and location of the lines 126 with respect to the clouds 122 instead of focusing on the imaging procedure itself.

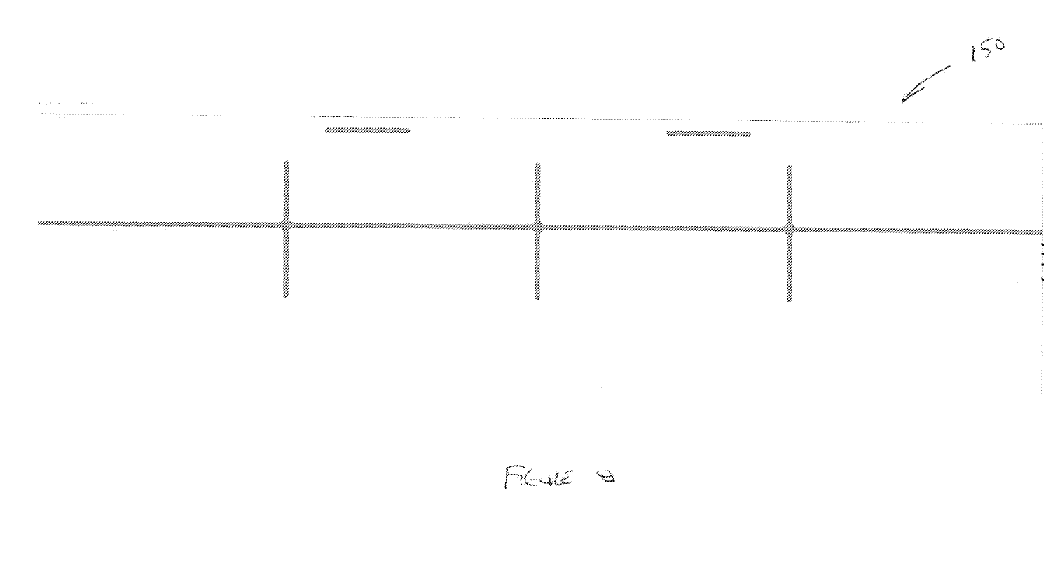

Referring to FIG. 7, elements 80 may be embodied to include a theme 140. The theme 140 may be another scenic design. For example, in the exemplary embodiment, the theme illustrated in FIG. 6 is a “Wood-grained Theme.” As such, the wood-grained theme 140 includes a visual representation of a wood-grained pattern 141 that is defined by a plurality of lines 142. As shown in FIG. 7, the wood-grained pattern 141 forms the background of the wood-grained theme 140. The wood-grained theme 140 may also include a geometric pattern 144 that is formed using a plurality of lines 146.

The geometric pattern lines 144 may include lines 146 having a first thickness and lines have a second thickness. Optionally, the geometric pattern 144 may include lines 146 having the same thickness. As discussed above, thicker lines are more easily observed by the patient 18, whereas thinner lines require the patient 18 to focus more on the theme 140. Accordingly, the patient 18 focuses less on the imaging procedure. It should be realized the weights and shapes of the lines 146 may be adjusted to generate a theme that is most beneficial in reducing the patient's motion. Thus, the lines 146 may be continuous, have a darker or lighter shade, and may be thicker or thinner. Moreover, in the exemplary embodiment, the lines 146 may be overlayed on at least a portion of the wood-grained pattern 141.

More specifically, the wood-grained pattern 141 functions as the background of the wood-grained theme 140 and the lines 146 function as the foreground of the wood-grained theme 140 such that the patient first observes the lines 146 and then upon further visual inspection observes the wood-grained pattern beneath the lines 146. The lines 146 may be the same color as a portion of the wood-grained pattern 141 or may be a different color. In operation, the patient's focus is therefore directed to first visually inspect the lines 146 and then visually inspect the size, shape and location of the lines 142 forming the wood-grained pattern 141 instead of focusing on the imaging procedure itself. Additionally, the wood-grained theme 140 may be drawn to include at least one random design element 148. The random design element may be embodied as a picture of a knot. In operation, the patient's focus is focused on locating the design element 148, e.g. the knots, instead of the imaging procedure itself.

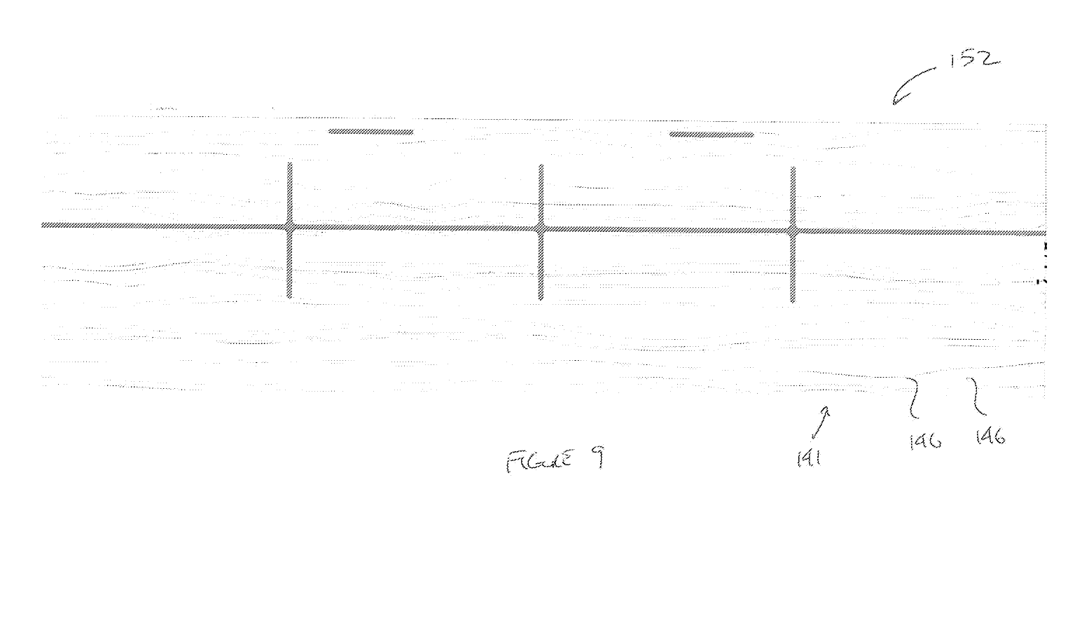

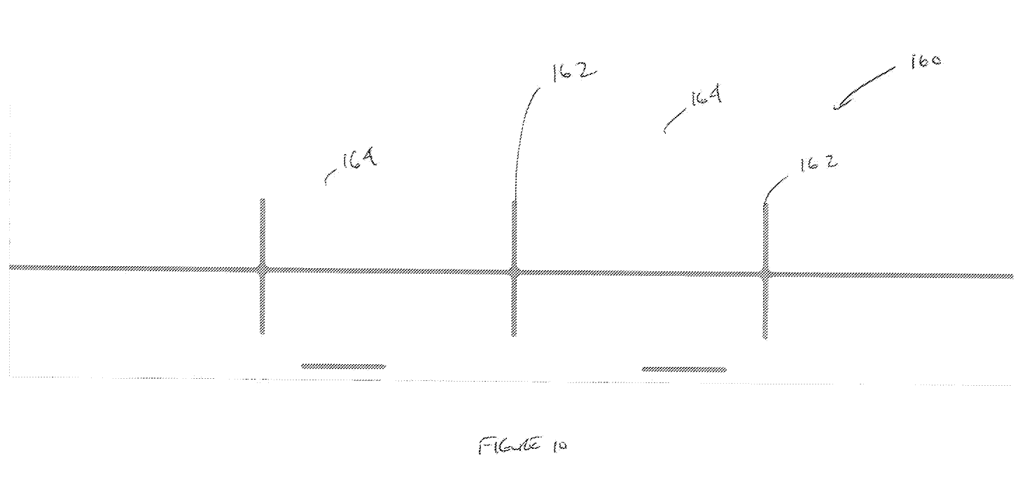

FIG. 8 illustrates another exemplary wood-grained theme 150. The wood-grained theme 150 is substantially similar to the wood-grained theme 140 shown in FIG. 7, with the design element 140, e.g. the knots, removed. FIG. 9 illustrates another exemplary wood-grained theme 152. The wood-grained theme 152 is substantially similar to the wood-grained theme 140 shown in FIG. 7. However, in this embodiment, the wood-grained pattern 141 is formed using lines 146 having various thicknesses.

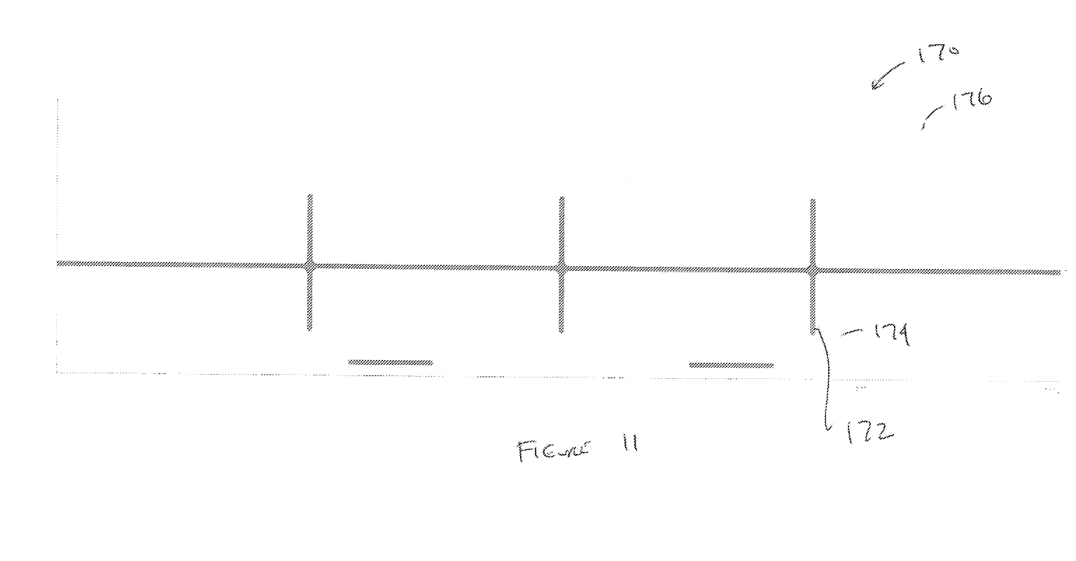

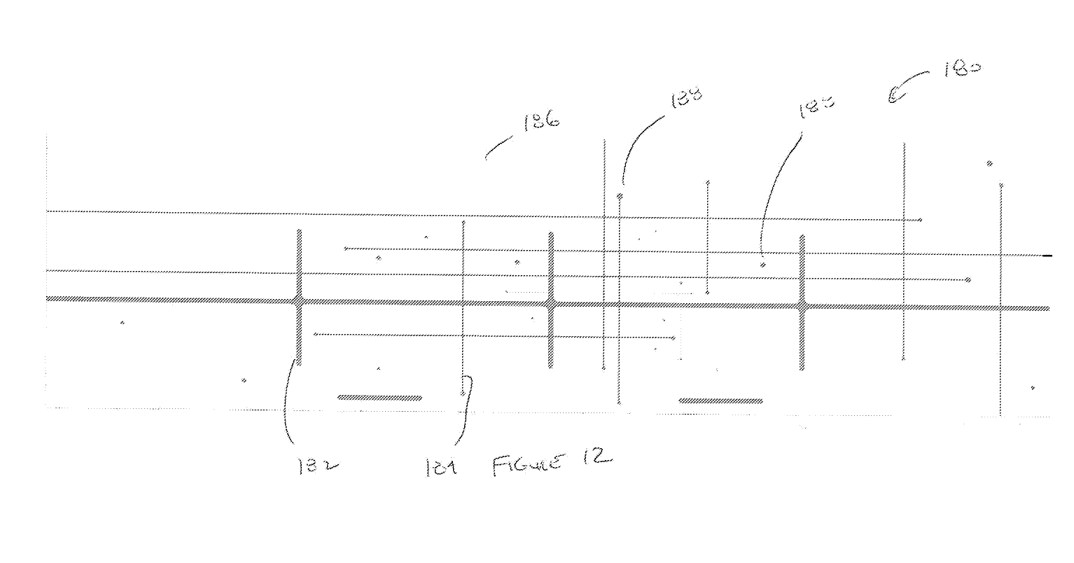

FIG. 10 is another exemplary theme 160 that may be used with the imaging system 10 to reduce patient motion. In the exemplary embodiment, the theme 160 is a geometric theme that is formed using a plurality of lines. The lines include a first plurality of lines 162 having a first thickness and a second plurality of lines 164 having a second thickness. As discussed above, the thicker lines 162 are more easily observed by the patient 18, whereas the thinner lines 164 require the patient 18 to focus more on the theme 160. Accordingly, the patient 18 focuses less on the imaging procedure. It should be realized the weights and shapes of the lines 162 and 164 may be adjusted to generate a theme that is most beneficial in reducing the patient's motion. Thus, the lines 162 and 164 may be continuous, have a darker or lighter shade, and may be thicker or thinner. In operation, the patient's focus is therefore directed to first visually inspect the lines 162 and then visually inspect the size, shape and location of the lines 164 with respect to the lines 162 122 instead of focusing on the imaging procedure itself FIG. 11 is another exemplary theme 170 that may be used with the imaging system 10 to reduce patient motion. In the exemplary embodiment, the theme 170 is a geometric theme that is formed using a plurality of lines. The lines include a first plurality of lines 172 having a first thickness, a second plurality of lines 174 having a second thickness, and a third plurality of lines 176 having a third thickness. As discussed above, the thicker lines 172 are more easily observed by the patient 18, whereas the thinner lines 176 require the patient 18 to focus more on the theme 170. Accordingly, the patient 18 focuses less on the imaging procedure. It should be realized the weights and shapes of the lines 172, 174, and 176 may be adjusted to generate a theme that is most beneficial in reducing the patient's motion. Thus, the lines 172, 174, and 176 may be continuous, have a darker or lighter shade, and may be thicker or thinner. In operation, the patient's focus is therefore directed to first visually inspecting the lines 172, then the lines 174, and finally the lines 176, due to shading, thickness, etc., instead of focusing on the imaging procedure itself.

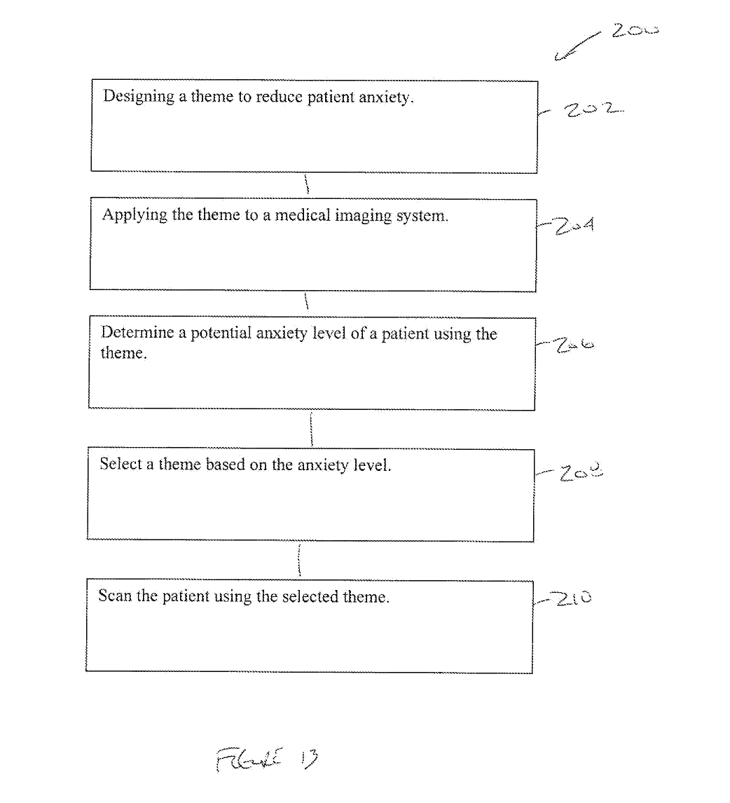

FIG. 12 is another exemplary theme 180 that may be used with the imaging system 10 to reduce patient motion. In the exemplary embodiment, the theme 180 is a geometric theme that is formed using a plurality of lines. The lines include a first plurality of lines 182 having a first thickness, a second plurality of lines 184 having a second thickness, and a third plurality of lines 186 having a third thickness. As discussed above, the thicker lines 182 are more easily observed by the patient 18, whereas the thinner lines 186 require the patient 18 to focus more on the theme 180. Accordingly, the patient 18 focuses less on the imaging procedure. It should be realized the weights and shapes of the lines 182, 184, and 186 may be adjusted to generate a theme that is most beneficial in reducing the patient's motion. Thus, the lines 182, 184, and 186 may be continuous, have a darker or lighter shade, and may be thicker or thinner. In operation, the patient's focus is therefore directed to first visually inspecting the lines 182, then the lines 184, and finally the lines 186, instead of focusing on the imaging procedure itself. Moreover, the theme 180 may also include a plurality of random design elements 188. The random design elements 188 may be embodied as, for example, dots. In operation, the patient is focused on locating the design element 188, i.e. the dots, instead of the imaging procedure itself.

FIG. 13 is a flowchart illustrating an exemplary method 200 for imaging a patient using a medical imaging system.

At 202, a theme is designed to reduce the patient's anxiety, and thus reduce the likelihood of patient movement during the imaging process and improve the image quality as a result of the decreased movement. A theme, as used herein, represents a unifying idea or subject that is implemented within the bore of the medical imaging system to reduce patient motion.

At 204, at least one imaging system is modified by applying the theme to at least a portion of the interior of the imaging system. The theme may be implemented directly on a bore cover of the imaging system. Optionally, the theme may be removably coupled to the bore cover of the imaging system.

At 206, a potential anxiety level of a patient is determined. In one embodiment, the patient's anxiety level may be determined based on a plurality of physiological parameters of the patient. For example, the anxiety level may be determined based on the patient's vital signs such as a patient's heart rate or breathing rate. The current heart and/or breathing rate may then be compared to previous heart rate or breathing rate data acquired from the same patient. A patient experiencing an increased heart or breathing rate is deemed to have an increased anxiety level. In another embodiment, the potential anxiety level of a patient is determined based on the patient's a priori knowledge of the medical imaging procedure. For example, one patient may have no knowledge of the imaging procedure and therefore have increased anxiety. Whereas another patient who has been previously imaged may have little or no anxiety. In one embodiment, the patient's anxiety is based on the patient's age. For example, a young patient may have no knowledge of the imaging procedure and therefore have increased anxiety. Whereas an older patient who has been previously imaged may have little or no anxiety.

At 208, a theme is selected based on the potential anxiety level. For example, assuming that the patient has relatively high anxiety level, an operator may determine that a first theme is best suited to reduce the anxiety level of the patient. Whereas another patient may be best suited for another theme. For example, a first patient may be imaged using a first imaging system having a first theme and a second different patient may be imaging using a second imaging system having a second theme. Optionally, the themes may be removable to enable a different theme to be installed based on the patient being imaged.

At 210, a medical imaging scan of the patient is performed. As discussed above, the medical scan may be performed using a computed tomography (CT) imaging system, a magnetic resonance imaging (MRI) system, a positron emission tomography (PET) imaging system, an ultrasound imaging system, an X-ray imaging system, and/or a single photon emission computed tomography (SPECT) imaging system.

Described herein is a medical imaging system, and methods that are configured to reduce the patient's anxiety while the patient being imaged and this reduce patient motion during imaging. A technical effect of the systems and methods described herein may reduce patient motion and thereby increase the quality of images. The exemplary embodiments described herein may also reduce the time required to perform patient imaging and increase the quality of the images. Specifically, various embodiments described herein include themes that are specifically designed to reduce the motion in patient's including younger patients. The imaging system includes a theme that represents a place, a pattern, or other design that is readily apparent to the patient being imaged. The themes are implemented using a plurality of theme-related elements. The theme-related elements may include scenic designs on the interior surface of the imaging system itself. The themes described herein are easily viewable by the patient being imaged and therefore focuses the patient's attention on the theme rather than on the imaging procedure. More specifically, the themes provide a distraction to focus the patient's attention during the imaging procedure. The distraction therefore reduces the patient's focus on the imaging procedure itself and therefore reduces the patient's anxiety level during the imaging procedure. As a result, motion of the patient may be reduced, thereby decreasing the time required to perform imaging and improving image quality.

It is to be understood that the above description is intended to be illustrative, and not restrictive. For example, the above-described embodiments (and/or aspects thereof) may be used in combination with each other. In addition, many modifications may be made to adapt a particular situation or material to the teachings of the various embodiments of the invention without departing from their scope. While the dimensions and types of materials described herein are intended to define the parameters of the various embodiments of the invention, the embodiments are by no means limiting and are exemplary embodiments. Many other embodiments will be apparent to those of skill in the art upon reviewing the above description. The scope of the various embodiments of the invention should, therefore, be determined with reference to the appended claims, along with the full scope of equivalents to which such claims are entitled. In the appended claims, the terms “including” and “in which” are used as the plain-English equivalents of the respective terms “comprising” and “wherein.” Moreover, in the following claims, the terms “first,” “second,” and “third,” etc. are used merely as labels, and are not intended to impose numerical requirements on their objects. Further, the limitations of the following claims are not written in means-plus-function format and are not intended to be interpreted based on 35 U.S.C. §112, sixth paragraph, unless and until such claim limitations expressly use the phrase “means for” followed by a statement of function void of further structure.

This written description uses examples to disclose the various embodiments of the invention, including the best mode, and also to enable any person skilled in the art to practice the various embodiments of the invention, including making and using any devices or systems and performing any incorporated methods. The patentable scope of the various embodiments of the invention is defined by the claims, and may include other examples that occur to those skilled in the art. Such other examples are intended to be within the scope of the claims if the examples have structural elements that do not differ from the literal language of the claims, or if the examples include equivalent structural elements with insubstantial differences from the literal languages of the claims.

What is claimed is:

1. A medical imaging system comprising:
a gantry having a bore extending therethrough;
a bore cover disposed within the bore; and
an element disposed on the bore cover, the element being viewable by a patient positioned within the bore to reduce patient motion.

2. A medical imaging system in accordance with claim 1, wherein the element creates a theme that forms a physical environment at least partially surrounding the patient, wherein the theme is presented in a manner to reduce an anxiety level of the patient being imaged.

3. A medical imaging system in accordance with claim 1, wherein the element is removably coupled to the bore cover.

4. A medical imaging system in accordance with claim 1, wherein the element is formed unitarily with the bore cover.

5. A medical imaging system in accordance with claim 1, wherein the element comprises at least one appliqué associated with the theme applied to the bore cover.

6. A medical imaging system in accordance with claim 5, wherein the bore cover has a first radius of curvature and the appliqué has the same radius of curvature.

7. A medical imaging system in accordance with claim 1, wherein the element comprises an ink printed on the bore cover to form the theme.

8. A medical imaging system in accordance with claim 1, wherein the theme comprises a cloud theme.

9. A medical imaging system in accordance with claim 8, wherein the cloud theme includes at least one random design element.

10. A medical imaging system in accordance with claim 1, wherein the theme comprises a cloud theme having at least one random design element.

11. A medical imaging system in accordance with claim 1, wherein the theme comprises a paisley theme including a plurality of random geometric shapes.

12. A medical imaging system in accordance with claim 1, wherein the theme comprises a cloud theme including a plurality of clouds and a geometric shape overlayed on the plurality of clouds.

13. A medical imaging system in accordance with claim 1, wherein the theme comprises a wood-grained theme including a wood-grained pattern and a geometric shape overlayed on the wood-grained pattern.

14. A medical imaging system in accordance with claim 1, wherein the theme comprises a wood-grained theme including a wood-grained pattern, a plurality of lines forming a geometric pattern, and at least one dot formed at an end of at least one of the lines.

15. A medical imaging system in accordance with claim 1, wherein the theme comprises a non-repeating geometric pattern.

16. A medical imaging system in accordance with claim 1 wherein the medical imaging system comprises at least one of a computed tomography (CT) imaging unit, a magnetic resonance imaging (MRI) unit, a positron emission tomography (PET) imaging unit, an ultrasound imaging unit, an X-ray imaging unit, and a single photon emission computed tomography (SPECT) imaging system.

17. A method of imaging a patient using a medical imaging system, said method comprising:
selecting a theme based on a potential anxiety level of a patient, wherein the theme is presented in a manner to reduce an anxiety level of the patient being imaged;
attaching the theme to a bore cover of the medical imaging system; and
performing a medical imaging scan of the patient using the medical imaging system.

18. The method of claim 17, further comprising attaching an appliqué associated with the theme to the bore cover.

19. The method of claim 17, further comprising printing the theme on the bore cover.

20. The method of claim 17, wherein the theme comprises a random paisley theme, a cloud theme, or a wood-grained theme.
